(19) United States
(12) Patent Application Publication
(10) Pub. No.: US 2022/0062065 A1
(43) Pub. Date: Mar. 3, 2022

(54) WATER-DISPERSIBLE FLUID ABSORBER

(71) Applicant: Symbioco Ltd, London (GB)

(72) Inventors: Aaron Mathews KOSHY, London (GB); Olivia Youngjoo AHN, London (GB)

(73) Assignee: Symbioco Ltd, London (GB)

(21) Appl. No.: 17/418,331

(22) PCT Filed: Dec. 27, 2019
(86) PCT No.: PCT/GB2019/053704

§ 371 Date: Jun. 25, 2021

(30) Foreign Application Priority Data
Dec. 27, 2018 (GB) 1821246.4
Sep. 10, 2019 (GB) 1913043.4

(51) Int. Cl. A61F 13/15; A61F 13/47

(52) U.S. Cl. CPC A61F 13/15577; A61F 13/15211; A61F 2013/53991; A61F 2013/15869; A61F 13/47

(57) ABSTRACT

A water-dispersible fluid absorber such as a sanitary pad includes a water-dispersible top layer; a water-dispersible absorbent layer; a leakproof layer of a polyvinyl alcohol; and a water-dispersible bottom layer. The leakproof layer is bonded to the top layer and the bottom layer by ultrasonic welding. A method of manufacturing a water-dispersible fluid absorber such as a sanitary pad includes assembling a water-dispersible top layer, a water-dispersible absorbent layer, a leakproof layer of a polyvinyl alcohol and a water-dispersible bottom layer; and bonding the leakproof layer to the top layer and the bottom layer by ultrasonic welding. A further water-dispersible fluid absorber includes a cellulose-based super absorbent material, an alginate-based super absorbent material, a chitosan-based super absorbent material, and/or a polyethylene glycol-based super absorbent material. A further water-dispersible fluid absorber comprises a thick portion formed to create a raised area under the perineum. A further water-dispersible fluid absorber includes a pressure sensitive adhesive arranged in a plurality of portions.

The present invention relates to a water-dispersible fluid absorber such as a sanitary pad.

Currently conventional personal hygiene products and other fluid absorbers are typically of non-flushable materials. The proliferation of such products is a stark problem for sanitation systems worldwide; non-flushable sanitary pad which are often flushed away (1.4 million sanitary pads and 2.5 million tampons flushed every day in the UK) are known to cause blockages in sewer or cistern systems which contribute to costs of £88 million a year (Sewer Misuse 2018, Water UK). Further, micro-plastics can be introduced in the water supply by non-biodegradable sanitary pads, which can persist in the environment. The present disclosure seeks to address these problems by presenting a fluid absorber such as a sanitary pad which is water-dispersible and biodegradable.

According to one aspect there is provided a water-dispersible fluid absorber such as a sanitary pad including: a water-dispersible top layer; a water-dispersible absorbent layer; and a leakproof layer of a polyvinyl alcohol (PVA); wherein the layers are bonded by ultrasonic welding.

Preferably the sanitary pad further includes a water-dispersible bottom layer, wherein the leakproof layer is bonded to the top layer and the bottom layer by ultrasonic welding.

By bonding the layers by ultrasonic welding a sufficiently strong bond can be achieved while maintaining water-dispersibility of the sanitary pad. Other bonding techniques, such as using adhesives or thermosealing, may have a negative impact on the water-dispersibility or provide inferior bond strength, may add another chemical in the process in the case of adhesives, or can cause brittleness and fragility of the treated area in the case of thermosealing. Preferably two or more of the layers are bonded by ultrasonic welding. PVA can provide sufficient water-dispersibility or water-solubility while being sufficiently waterproof for use as a leakproof layer. PVA can also enable good bonding by ultrasonic welding. Preferably the sanitary pad is biodegradable.

For water-dispersibility and biodegradability the top layer may be a cellulosic nonwoven fabric, preferably a wet-laid and/or spunlace and/or semi-hydrophobic cellulosic nonwoven fabric. A wet-laid fabric can enable good water-dispersibility. A spunlace fabric can enable good water-dispersibility. A semi-hydrophobic fabric can enable good wicking of moisture and provide a favourable dry feel. The top layer may be of wood pulp and/or hydrophobic cellulosic fibres to provide a water-dispersible and semi-hydrophobic top layer. The top layer may comprise a PVA or carboxymethylcellulose binder for good water-dispersibility and biodegradability. The top layer may be a wet-laid spunlace fabric.

The top layer and optionally the absorbent layer and optionally the support layer and optionally the acquisition distribution layer may include embossments. The top layer may be weakly bonded to the leakproof layer at the embossments. This can reduce lateral movement of portions of the absorbent layer. Such a weak bond is preferably severable without damaging the layers.

For water-dispersibility and biodegradability the absorbent layer may include cellulosic fibres, preferably wood pulp fibres. The absorbent layer may include a blend of wood pulp fibres and viscose rayon fibres. The absorbent layer may be of wood pulp fibres.

The absorbent layer may include a super absorbent polymer. For biodegradability the super absorbent polymer is preferably a cellulose-based material. The super absorbent polymer may be a sodium carboxymethylcellulose. The super absorbent polymer may be an alginate material. The super absorbent polymer may be a chitosan material. The super absorbent polymer may be a polyethylene glycol material. The super absorbent polymer may be a hybrid material including two or more of the aforementioned polymers.

The super absorbent polymer may be bonded to an adjacent material or layer. The super absorbent polymer may be bonded to the leakproof layer. The super absorbent polymer may be bonded without an additional adhesive.

Preferably the leakproof layer is bonded to the top layer. This arrangement can permit sufficient bonding due to the suitability of PVA for ultrasonic welding. Preferably the absorbent layer is enclosed by the leakproof layer and the top layer.

For support the sanitary pad may further include a water-dispersible support layer. This can enable a more robust sanitary pad, for example for use when asleep, or for absorbing a relatively large amount of fluid. The support layer is preferably arranged between the absorbent layer and the leakproof layer. For water-dispersibility and biodegradability the support layer is preferably a cellulosic nonwoven fabric, preferably a wet-laid and/or spunlace cellulosic nonwoven fabric.

For support the sanitary pad may further include a water-dispersible bottom layer. The bottom layer may be a water-dispersible and biodegradable nonwoven material. The bottom layer may be a cellulosic nonwoven fabric, preferably a wet-laid and/or spunlace and/or semi-hydrophobic cellulosic nonwoven fabric. The bottom layer may be of wood pulp and/or hydrophobic cellulosic fibres. The bottom layer may be a wet-laid spunlace fabric. The leakproof layer is preferably arranged between the water-dispersible top layer and the water-dispersible bottom layer. The leakproof layer is preferably simultaneously bonded to the top layer and the bottom layer by ultrasonic welding. The bottom layer may extend beyond the top layer to form two opposing tabs extending from the main body of the sanitary pad.

For enhanced performance the sanitary pad may further include a water-dispersible acquisition distribution layer. The acquisition distribution layer is preferably arranged between the top layer and the absorbent layer. The acquisition distribution layer is preferably a cellulosic nonwoven fabric, preferably a wet-laid and/or spunlace cellulosic nonwoven fabric. To improve lateral distribution of fluid the acquisition distribution layer is preferably with embossments.

The leakproof layer may be a polyvinyl alcohol film that is preferably between 20 and 150 μm thick, more preferably between 30 and 90 μm thick, more preferably between 30 and 65 μm thick. This can provide sufficient strength and stability while remaining sufficiently water-soluble and water-dispersible. The leakproof layer may comprise a hydrophobic surface. The leakproof layer may be laminated to the bottom layer and/or co-extruded with the bottom layer.

For increasing the bond strength the leakproof layer may be folded over the top layer to encase partially a portion of the top layer. This can form a double seam.

For additional bonding the sanitary pad may further include an adhesive arranged between two or more of the layers. For additional bonding the sanitary pad may further include a bond formed by thermosealing.

For attachment to an undergarment the sanitary pad may further include a pressure sensitive adhesive. The pressure sensitive adhesive (PSA) may be arranged in a plurality of portions. This can enable sufficient adhesion while limiting the individual portions in size. Larger portions of PSA can reduce the water-dispersibility of the sanitary pad. Each portion may have an area of 5 to 250 mm², preferably 25 to 100 mm². Each portion may have an area of 1 to 100 mm², preferably 1 to 25 mm². Each portion may have a diameter or edge length of 0.5 to 10 mm, preferably 1 to 5 mm. The pressure sensitive adhesive may be arranged in 5 to 100 portions, or in 10 to 60 portions, or in 30 to 500 portions, or 30 to 2000 portions. The pressure sensitive adhesive may be arranged with 1 to 20 mm between portions, preferably 1 to 10 mm. For good adhesion the pressure sensitive adhesive portions may be distributed regularly over the sanitary pad, preferably in a grid pattern. The pressure sensitive adhesive (PSA) may be arranged to include a plurality of perforations. Perforations can assist with water-dispersibility.

For convenience the sanitary pad may further include a water-dispersible (or water-soluble or biodegradable) release liner to protect the pressure sensitive adhesive. The release liner may be of a water-dispersible paper-based material. The release liner may be of a water-soluble PVA material.

For good attachment the sanitary pad may be shaped with two opposing tabs extending from the main body of the sanitary pad. The tabs may be narrower at the junction to the main body of the sanitary pad than at the widest portion of the tabs. This can reduce the length of the fold when the tabs are folded back for attachment. The fold can be prone to damage in use. Each tab may be substantially trapezoidal.

The sanitary pad may comprise a thick portion, preferably in a central portion of the sanitary pad. The thick portion may be formed to create a raised area under the perineum. The thick portion can help reduce back leaking in use, in particular for use while sleeping.

According to another aspect there is provided a method of manufacturing a water-dispersible fluid absorber such as a sanitary pad including: assembling a water-dispersible top layer, a water-dispersible absorbent layer and a leakproof layer of a polyvinyl alcohol; and bonding the layers by ultrasonic welding. By bonding the layers by ultrasonic welding a sufficiently strong bond can be achieved while maintaining water-dispersibility of the sanitary pad. The sanitary pad may be as aforementioned.

The method may further comprise including a water-dispersible bottom layer and bonding the leakproof layer to the top layer and the bottom layer by ultrasonic welding.

The method may further comprise embossing one or more layers by ultrasonic embossing. The ultrasonic embossing and the ultrasonic welding may be performed simultaneously and/or by the same tool. The ultrasonic embossing is preferably of the top layer and optionally at least partially of the absorbent layer. The ultrasonic welding is preferably of the top layer and the leakproof layer, at least.

The method may further comprise enclosing the absorbent layer between the leakproof layer and the top layer.

The method may further comprise bonding a super absorbent polymer to an adjacent material or layer. The super absorbent polymer may be bonded to the leakproof layer. The super absorbent polymer may be bonded by moistening the leakproof layer. The super absorbent polymer may be electrostatically bonded.

For additional bonding the method may further comprise additionally bonding two or more of the layers with an adhesive. The method may further comprise additionally bonding two or more of the layers by thermosealing.

The method may further comprise providing a patterned layer of a pressure sensitive adhesive with rotogravure printing.

According to another aspect there is provided a water-dispersible fluid absorber such as a sanitary pad including: a water-dispersible top layer; a water-dispersible absorbent layer; and a water-dispersible (or water-soluble) leakproof layer; wherein the absorbent layer comprises a cellulose-based super absorbent material and/or an alginate-based super absorbent material a chitosan-based super absorbent material, and/or a polyethylene glycol-based super absorbent material.

By providing an absorbent layer comprising such a super absorbent material biodegradability and good absorption can be achieved. The absorbent layer may be as aforementioned. The sanitary pad may be as aforementioned.

The super absorbent polymer may be bonded to an adjacent material or layer. The super absorbent polymer may be bonded to the leakproof layer. The super absorbent polymer may be bonded to a portion of the absorbent layer. The super absorbent polymer may be bonded in the absence of an additional adhesive.

According to another aspect there is provided a water-dispersible fluid absorber such as a sanitary pad including: a water-dispersible top layer; a water-dispersible absorbent layer; and a water-dispersible (or water-soluble) leakproof layer; wherein the sanitary pad comprises a thick portion formed to create a raised area under the perineum.

By providing a thick portion formed to create a raised area under the perineum back-leaking in use can be reduced, in particular for use while sleeping. The thick portion may be as aforementioned. The sanitary pad may be as aforementioned.

According to another aspect there is provided a water-dispersible fluid absorber such as a sanitary pad including: a water-dispersible top layer; a water-dispersible absorbent layer; a water-dispersible (or water-soluble) leakproof layer; and a pressure sensitive adhesive; wherein the pressure sensitive adhesive is arranged in a plurality of portions.

By providing the pressure sensitive adhesive in a plurality of portions good adhesion can be enabled while maintaining water-dispersibility. The portions may be as aforementioned. The sanitary pad may be as aforementioned.

Each portion may have an area of 5 to 250 mm², preferably 25 to 100 mm². Each portion may have an area of 1 to 100 mm², preferably 1 to 25 mm². Each portion may have a diameter or edge length of 0.5 to 10 mm, preferably 1 to 5 mm. The pressure sensitive adhesive may be arranged in 5 to 100 portions, or in 10 to 60 portions, or in 30 to 500 portions, or 30 to 2000 portions. The pressure sensitive adhesive may be arranged with 1 to 20 mm between portions, preferably 1 to 10 mm. For good adhesion the pressure sensitive adhesive portions may be distributed regularly over the sanitary pad, preferably in a grid pattern. The pressure sensitive adhesive (PSA) may be arranged to include a plurality of perforations. Perforations can assist with water-dispersibility.

According to another aspect there is provided a water-dispersible fluid absorber such as a sanitary pad including: a water-dispersible top layer; a water-dispersible absorbent layer; and a water-dispersible (or water-soluble) leakproof layer. The sanitary pad may include one or more of the features as aforementioned.

According to another aspect there is provided a water-dispersible fluid absorber including: a water-dispersible top layer; a water-dispersible absorbent layer; and a water-dispersible (or water-soluble) leakproof layer. The fluid absorber may include one or more of the features as aforementioned. The fluid absorber may be a sanitary pad, an incontinence pad, a wound dressing, a diaper or a cleaning pad. The leakproof layer may be of a polyvinyl alcohol (PVA). The layers may be bonded by ultrasonic welding. The absorbent layer may comprise a cellulose-based super absorbent material. The fluid absorber may comprise a thick portion formed to create a raised area under the perineum. The fluid absorber may comprise a pressure sensitive adhesive. The pressure sensitive adhesive may be arranged in a plurality of portions.

Any apparatus feature as described herein may also be provided as a method feature, and vice versa.

Any feature in one aspect of the invention may be applied to other aspects of the invention, in any appropriate combination. In particular, method aspects may be applied to apparatus aspects, and vice versa. Furthermore, any, some and/or all features in one aspect can be applied to any, some and/or all features in any other aspect, in any appropriate combination.

It should also be appreciated that particular combinations of the various features described and defined in any aspects of the invention can be implemented and/or supplied and/or used independently.

As used herein, the terms ‘water-dispersible’, ‘water-soluble’ and ‘biodegradable’ preferably mean compatible with the UK SNAP protocol for testing disintegration in drainline and/or compatible with the UK Fine-to-Flush protocol for testing disintegration in drainline and/or compatible with the INDA EDANA “Guidelines for Assessing the Flushability of Disposable Nonwoven Products” and/or compatible with the IWSFG Slosh Box Disintegration Test and/or compatible with one of the OECD 301 tests.

As used herein, the term ‘sanitary pad’ preferably means a pad suitable for attachment to an undergarment and suitable for absorbing a flow of blood from the vagina. A sanitary pad may also be referred to as a sanitary napkin, sanitary towel or menstrual pad. As used herein the term ‘sanitary pad’ preferably includes maternity pads, panty liners, and night pads. The term ‘sanitary pad’ may include incontinence pads and/or haemorrhoid pads.

These and other aspects of the present invention will become apparent from the following exemplary embodiments that are described with reference to the following figures in which:

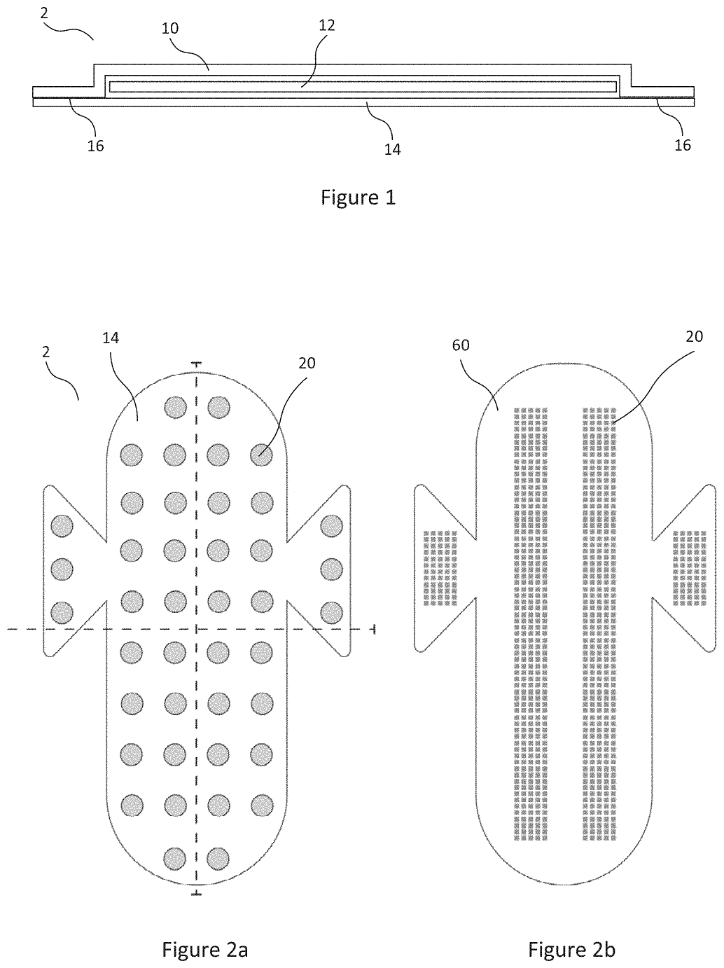

FIG. 1 shows a sectional view of a sanitary pad;

FIG. 2a shows a bottom view of a sanitary pad;

FIG. 2b shows a bottom view of another sanitary pad;

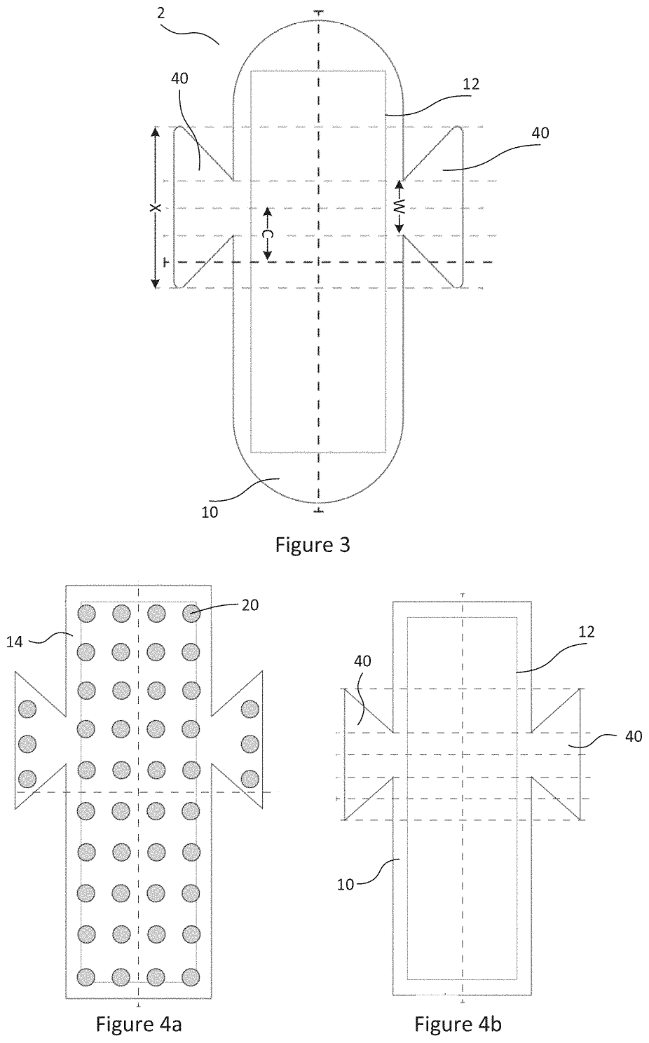

FIG. 3 shows a top view of the sanitary pad of FIG. 2a;

FIG. 4a shows a bottom view of another sanitary pad;

FIG. 4b shows a top view of the sanitary pad of FIG. 4a;

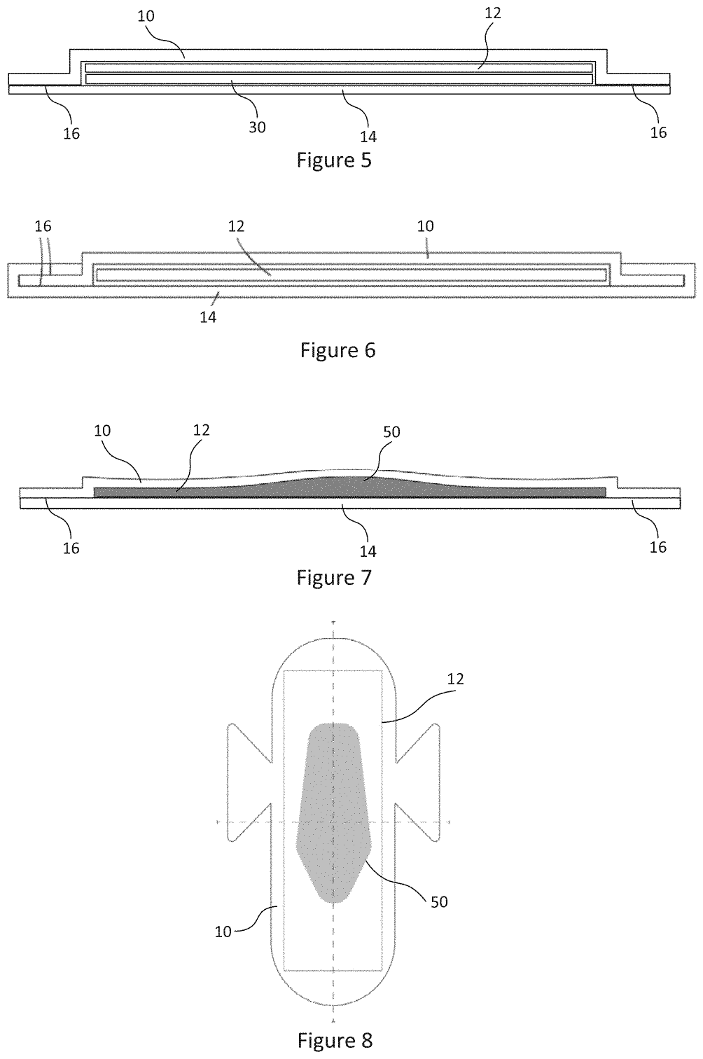

FIG. 5 shows a sectional view of a sanitary pad with a support layer;

FIG. 6 shows a sectional view of a sanitary pad with a double seam;

FIG. 7 shows a sectional view of a sanitary pad with a thick portion;

FIG. 8 shows a top view of the sanitary pad of FIG. 7;

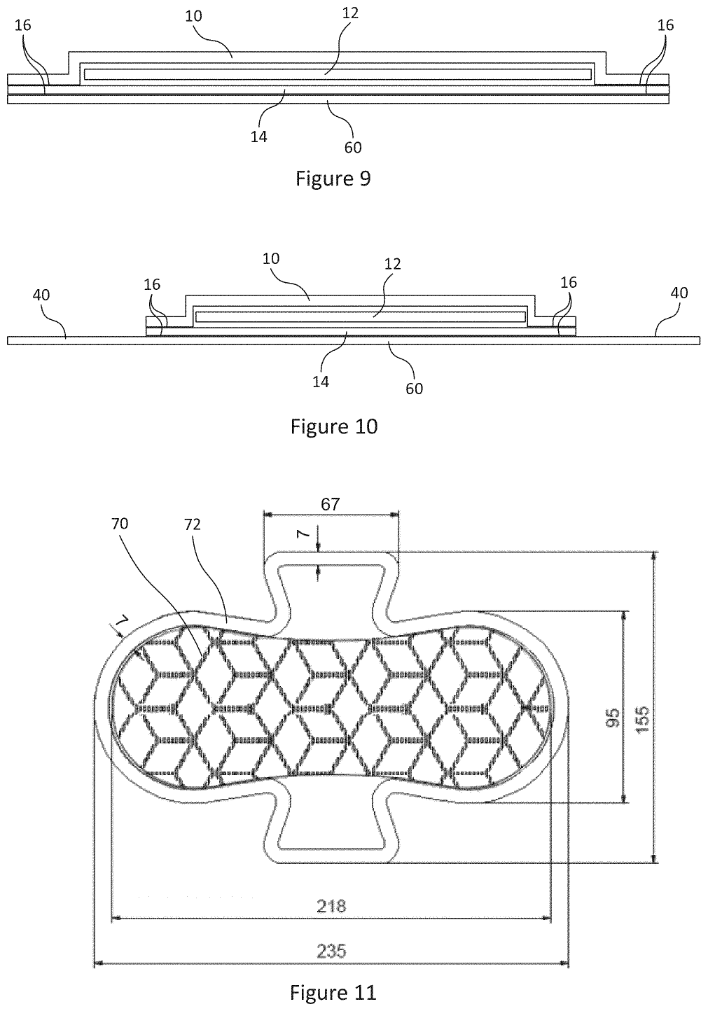

FIG. 9 shows a sectional view of a sanitary pad with a bottom layer;

FIG. 10 shows a sectional view of a sanitary pad with a bottom layer forming opposing tabs;

FIG. 11 shows a top view of a tool for forming a sanitary pad with embossments in the top layer;

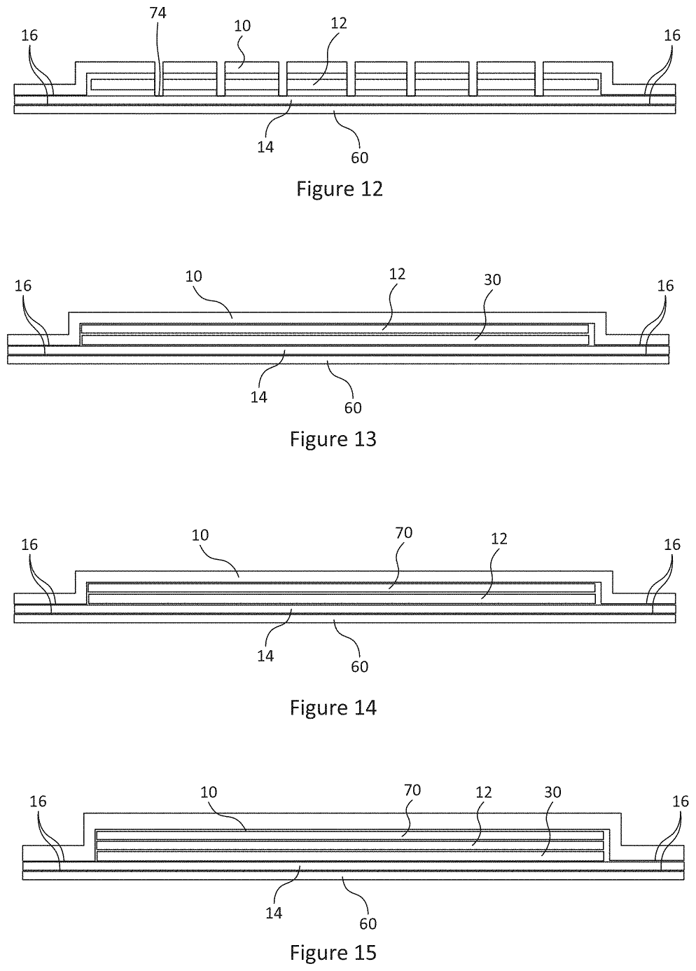

FIG. 12 shows a sectional view of a sanitary pad with embossments;

FIG. 13 shows a sectional view of a sanitary pad with a bottom layer and a support layer;

FIG. 14 shows a sectional view of a sanitary pad with a bottom layer and an acquisition distribution layer; and

FIG. 15 shows a sectional view of a sanitary pad with a bottom layer, an acquisition distribution layer and a support layer.

FIG. 1 shows an example of a sanitary pad 2. An absorbent layer 12 is enclosed between a top layer 10 and a leakproof layer 14. The top layer 10 is of a non-woven cellulosic material. The absorbent layer 12 is of an absorbent material. The leakproof layer 14 is of a poly vinyl alcohol (PVA). The top layer 10 and the leakproof layer 14 are bonded at the edge 16 of the sanitary pad 2 by ultrasonic bonding.

The sanitary pad 2 is able to absorb bodily fluids and subsequent to use can disperse well in water at ambient temperatures. It is recognised that finding a combination of materials and bonding them appropriately, such that the requirement to absorb whilst remaining stable and also the requirement to disperse are fulfilled, is a challenge. The sanitary pad 2 can provide a solution that fulfils these conflicting requirements.

The top layer 10 has high fluid permeability to ensure that body fluid is able to pass quickly through the top layer 10 to the interior of the sanitary pad 2. It is desirable for body fluid to be transferred rapidly away from the top layer 10; therefore a material with a high wicking value is desirable for the top layer 10. The top layer 10 is of a cellulosic non-woven fabric. The cellulosic fibres may for example be natural cellulose fibres or manufactured cellulose fibres, such as rayon fibres including viscose rayon. Examples of suitable cellulosic fibres include those available under the trade name Hydraspun Dispersible, Hydraspun Plus; or Viloft or Orlea from Kelheim Fibres. Such fabrics are typically of a non-woven fibre and may include a water-soluble binder such as PVA or carboxymethylcellulose (CMC), or may be formed without binder. Other suitably dispersible top layers may be used. A suitable top layer may for example have a mass per unit area of 25-100 grams per square metre (gsm), 40-80 gsm, 50-75 gsm, or approximately 50 gsm.

In an example short-cut Viloft viscose rayon fibres are processed into a non-woven fabric through a wet-laid spunlace process. In another example a wet-laid semi-hydrophobic top layer is substantially of wood pulp and hydrophobic cellulosic fibre (such as Olea hydrophobic viscose rayon fibres from Kelheim Fibres). A wet-laid material is observed to have superior water-dispersibility. A semi-hydrophobic material can provide a favourable dry feel as the moisture is wicked to the absorptive core rather than remaining on the surface of the sanitary pad.

Advantageously the top layer is a wet-laid spunlace (WLS). Spunlace (also referred to as hydroentanglement) is formed by a mechanical process that requires no binder (PVA or CMC). Dispersibility can be higher with WLS than with air-laid/dry-laid fabric as no binder is used. WLS material may have a relatively low wet strength, which may be acceptable for use in a sanitary pad or similar where the material goes from dry to wet and is not subject to excessive shear force during use (until exposed to the hydraulic action in a toilet).

The absorbent layer 12 is formed of wet-laid non-woven fibres, preferably with a super absorbent polymer included to enhance the absorbency. To enable biodegradability the fibres are preferably cellulosic. The cellulosic fibres may for example be natural cellulose fibres such as wood pulp fibres, or manufactured cellulose fibres, such as rayon fibres including viscose rayon. The absorbent layer 12 may include a blend of different fibres, for example a blend of both wood pulp fibres and viscose rayon fibres. This can provide good pliability and stability. Examples of suitable cellulosic fibres include those available under the trade name Danufil, Galaxy or Viloft from Kelheim Fibres or others.

In an example the absorbent layer 12 is formed by pressing loose wood pulp fibres into shape.

The super absorbent polymer may be a partially cross-linked cellulose-based material such as a sodium carboxymethylcellulose. Cellulose-based super absorbents are biodegradable and food safe, unlike other super absorbent polymers conventionally used (such as polyacrylamides or polyacrylates). The super absorbent polymer may be a cellulose-based material such as those available under the trade name of Aquasorb® cellulose gum from Ashland. The super absorbent polymer may be another biodegradable polymer, such as an alginate material, a chitosan, a polyethylene glycol, or a hybrid material such as a cellulose-chitin hybrid or a cellulose-polyethylene glycol hybrid. The super absorbent is typically provided as a powder but may be provided in another form, for example as a film, optionally bonded to another material.

If a super absorbent polymer is included in the absorbent layer 12, then the absorbent layer 12 may be formed of super absorbent polymer homogenously mixed with the fibres. Alternatively the super absorbent polymer may be provided in a layer, whether as a film or as granular or powder material. For example, a layer of super absorbent polymer may be arranged in the interior of the absorbent layer 12, for example sandwiched between two layers of fibres of the absorbent layer 12. In another example a layer of super absorbent polymer is arranged at a boundary of the absorbent layer 12, for example between a layer of fibres of the absorbent layer 12 and the leakproof layer 14 (or another layer such as a support layer).

The super absorbent polymer may be bonded to another material, for example fibres, for example at a top or a bottom of the absorbent layer 12. The super absorbent polymer may be bonded to a layer adjacent the absorbent layer, for example to the leakproof layer 14 or to a support layer. Bonding the super absorbent polymer can prevent it from slipping relative to other materials and layers, and facilitate manufacture of the pad.

In an example super absorbent polymer is bonded to the leakproof layer 14. Although a water-dispersible adhesive can be used, for water-dispersibility of the sanitary pad and ease of manufacture avoidance of an additional water-dispersible adhesive is preferred.

For example, the leakproof layer 14 is exposed to moisture or humidity, e.g. by exposure to mist or in a humidifying chamber, causing the surface of the leakproof layer 14 (in particular a PVA leakproof layer 14) to become tacky enough to adhere a super absorbent polymer to the leakproof layer 14. In another example static electricity between the leakproof layer 14 and the super absorbent polymer cause the super absorbent polymer to adhere to the leakproof layer 14. Once the super absorbent polymer is bonded to the leakproof layer 14, fibres of the absorbent layer 12 can be arranged on the super absorbent polymer and a pneumatic press can apply pressure to the fibres of the absorbent layer 12 to form absorbent layer 12.

The leakproof layer 14 forms a fluid impermeable layer. On exposure to body fluid or body moisture the structurally integrity of the water-dispersible fluid absorber is not adversely affected. The leakproof layer 14 provides a barrier to fluids in the absorbent layer 12. The leakproof layer 14 is in an example a polyvinyl alcohol (PVA) such as those available under the trade name Solublon or Hydropol. The leakproof layer 14 is in an example 35, 40, 50, 75 or 90 μm thick. The polyvinyl alcohol of the leakproof layer 14 is a water-soluble PVA or a water-dispersible PVA. The film may be smooth. A PVA film is typically composed of PVA copolymer. The melting point of a thermoplastic PVA film is typically 140° C.-200° C.

An example of the properties of a 50 μm water-soluble thermoplastic PVA film that is suitable as a leakproof layer 14 are (at 23° C.-27° C. and 50%-55% relative humidity):

| Characteristic | Typical Values | | Measurement protocol |
|---|---|---|---|
| Elongation at Break | 320% | | ASTM D882 |
| Tensile Strength | 32 | N/mm² | ASTM D882 |
| Ultimate Modulus | 170 | MPa | ASTM D882 |
| Impact Strength | 1400 | g | ASTM D1709 |
| Disintegration time at 70° C. | 12 | s | IS 16154:2014 |
| Dissolution time at 70° C. | 39 | s | IS 16154:2014 |

| | | |
|---|---|---|
| Moisture Content | 10% | IS 16154:2014 |

An example of the properties of another water-soluble thermoplastic PVA film (here a film of modified co-polymers based on vinyl acetate hydrolysed monomers designed to be warm soluble, available under the trade name Aquapak Hydropol 33100) that is suitable as a leakproof layer 14 is:

| | | | Measurement |
|---|---|---|---|
| Characteristic | 25 μm film | 35 μm film | protocol |
| Tensile Strength | 79 MPa (machine | | ASTM D882 |
| | direction) | | |
| | 66 MPa (cross | | |
| | direction) | | |
| Elongation | 226% (machine | | ASTM D882 |
| | direction) | | |
| | 139% (cross | | |
| | direction) | | |
| Dart puncture | 272 g | | ASTM 1709 |
| Oxygen transmission rate | | 0.0581 | ISO 1505-2 |
| (0% RH, 23° C.) | | cc/m²/24 hr | |

Above properties are given for a 25 μm film or a 35 μm film, but other thicknesses, for example 40, 50 or 75 μm, may be selected.

An example of the properties of another water-soluble thermoplastic PVA film (here a film designed to be cold soluble—designed to dissolve at temperatures above 1° C.—available under the trade name Aicello SOLUBLON® PT B) that is suitable as a leakproof layer 14 are (at 23° C. and 50% relative humidity):

| | | | | | Measurement |
|---|---|---|---|---|---|
| Characteristic | 75 μm film | | 90 μm film | | protocol |
| Tensile Strength | 44 | MPa | 44 | MPa | ASTM D882 |
| Elongation | 420% | | 430% | | ASTM D882 |
| Young's Modulus | 31 | MPa | 32 | MPa | ASTM D882 |
| Disintegration time at 20° C. | 28 | s | 45 | s | IS 16154:2014 |
| Dissolution time at 20° C. | 49 | s | 72 | s | IS 16154:2014 |

Other water-soluble thermoplastic PVA films, including films designed to be warm soluble (for instance designed to dissolve at temperatures above 30° C. or above 40° C. or above 45° C.), e.g. at thicknesses of 35-75 μm, are also suitable. Thinner films can provide better flexibility of the sanitary pad, and warm soluble films can provide better stability (e.g. against perspiration) than cold soluble films at reduced thickness. It is observed that the dissolution time for a relatively thin (e.g. 35-70 μm) warm-soluble film is longer than the dissolution time for a relatively thick (e.g. 90 μm) cold-soluble film, but both perform adequately to comply with the fine-to-flush guidelines and are sufficiently water-disintegrable/water-soluble. The film thickness is selected for the film to remain sufficiently stable under the influence of shear forces during use.

The top layer 10 and the leakproof layer 14 are bonded at the edge 16 of the sanitary pad 2 by ultrasonic bonding. The layers may be selected such that they are smooth in the area of the bond for good bond quality; textured or embossed surfaces in the bond area can reduce the bond strength.

Ultrasonic bonding can enable a bond between the layers that is sufficiently strong for use, and yet is readily enough fluid dispersible to enable flushing of the sanitary pad. While a suitable water-soluble adhesive can be used, it is observed that either the adhesion is unsatisfactory and the layers can separate, or the water-dispersibility of the pad is poor. In particular PVA is observed to form a good bond with water-dispersible cellulosic layers by ultrasonic bonding.

Ultrasonic bonding can join materials by way of frictional heat generated from high frequency (ultrasonic) mechanical motion. Under application of pressure and the frictional heat the PVA in the joint area can melt to create a bond between layers. It is observed that bonds formed by thermosealing (where heat is applied without ultrasound) are more brittle and inflexible, and are at greater risk of damage in use than bonds formed by ultrasonic bonding. Thermosealing (or related thermal embossing) can supplement, but not replace, ultrasonic bonding.

In a simple example ultrasonic bonding is implemented with a suitably shaped horn or a sonotrode that can be pressed against an anvil on which the layers to be bonded can be arranged to cut and weld the layers together. For example, for a rotationally symmetrical sanitary pad shape a horn or sonotrode is shaped so as to seal a half of a pad and then the substrate is rotated to seal the other half. As the surface area of the bond surface is large, for satisfactory bonding only half the pad is bonded per sonotrode and stroke. In another example two horns or sonotrodes are provided that together are shaped to provide a seal around the pad. Pressing both horns or sonotrodes down onto the unbonded layers permits bonding of the layers to form a pad in a single work stroke, without repositioning the pad. While the horn presses onto the layers, the central area of the pad and in particular the absorbent layer can be flattened and made more uniformly thick.

Suitable parameters for each sonotrode are for example:

Frequency—20 KHz

Energy—3000 Watt

Cycle Time—5 seconds

Amplitude—20 μm

Force—3000 Newton

Fixture Make—Aluminium (for ease of manufacturing) or titanium (for robustness)

In another example the ultrasonic bonding is by a rotational ultrasonic bonding process with a continuous drum or wheel that cuts and welds the layers together.

In some examples the ultrasonic bonding is the sole means of adhering the layers together to form the sanitary pad, without use of an adhesive (an adhesive being a substance other than the layers applied to bind the layers together). In other examples the ultrasonic bonding is supplemented by use of an adhesive, typically a water-based water-soluble PVA adhesive, to adhere different layers together during the manufacturing procedure. This may be appropriate in the case of pads such as a night pad or in an incontinence pad. Suitably water-soluble adhesives are observed to dissolve prematurely during use, whereas the ultrasonic bonds form a stronger bond, strong enough for the bonds to remain intact in use; therefore the adhesive is not considered sufficient on its own. In other examples the ultrasonic bonding is supplemented by use of thermosealing or thermal embossing, for example to emboss a pattern into one or more layers.

For attachment of the sanitary pad 2 to undergarments, pressure sensitive adhesive is present on the external surface of the sanitary pad.

FIG. 2a shows an example where the adhesive 20 is present in a regular pattern on the full area of the sanitary pad. In the illustrated example the adhesive 20 is in a round-shaped dot matrix pattern. In an example the dots each have a diameter of up to 10 mm and an area of approximately 75 mm².

FIG. 2b shows an example where the adhesive 20 is present in a dot pattern arranged in four strips: two strips along the length of the main body and one strip on each tab. Each strip is 20-30 mm wide. Each strip contains square-shaped adhesive portions in a grid pattern. In the illustrated example the square-shaped adhesive dots each have an edge length of 2.8 mm and a gap of 1.5 mm between dots (for a 20 mm wise strip) or an edge length of 4.2 mm and a gap of 2.2 mm between portions (for a 30 mm wise strip).

In other examples the adhesive is arranged in a pattern (e.g. grid pattern, hexagonal pattern, or other decorative or figurative pattern) of dots (e.g. square-shaped, diamond-shaped, triangular-shaped, round-shaped or irregular-shaped dots), each dot having a diameter or edge length of 1-5 mm, with a gap of 1-10 mm between dots. The distribution of the adhesive in relatively small portions over a relatively large area permits use of an adhesive with moderate water solubility without substantially reducing the water-dispersibility of the sanitary pad. On contact with water, the sanitary pad retains its ability to disperse sufficiently. Providing the adhesive in relatively small portions can also increase the surface area to accelerate biodegradation.

In another example the pressure sensitive adhesive is a film, with or without perforations. Perforations can improve the hydraulic dispersibility of the adhesive, but may introduce mechanical weakness. Sufficient dispersibility can be achieved without perforations. A transfer tape adhesive such as a 10-35 gsm water-based cold water soluble pressure sensitive tape adhesive with an average peel strength of 5N can provide a suitable adhesive film. Hot melt adhesives are not sufficiently biodegradable and therefore a transfer tape adhesive is preferred.

In another example a pressure sensitive adhesive is applied in a pattern to a release liner (such as a silicone coated paper or a water-dispersible liner) by way of rotogravure printing. In an example an adhesive suitable for rotogravure printing is a water-based, cold water soluble, pressure sensitive adhesive emulsion with a viscosity around 250 cps (measured with a Brookfield viscometer at 2 rpm and at 50 rpm at 20° C.), a solid content of around 50% and a specific gravity of around 1.05 g/ml. The patterned adhesive is sandwiched between the surface of the sanitary pad that is intended for undergarment attachment and the release liner in order to affix the adhesive to the sanitary pad. The release liner can be included as is in the sanitary pad, or it can be replaced by another release liner, e.g. to replace a silicone-based liner with a more water-dispersible liner for the final product.

A water-dispersible release liner such as a paper-based liner or a PVA liner may be used to protect the adhesive 20. The release liner is removed prior to the use of the sanitary pad.

A suitable water-dispersible release liner may be a hydrophobic coated PVA film and/or an embossed PVA film.

An example of the properties of a 80 μm thick paper-based liner (here a paper that includes a heat seal coating as is available under the trade name SmartSolve 3150A) that is suitable as a water-dispersible release liner are (at 23° C. and 50% relative humidity):

| Characteristic | Typical Values | | Measurement protocol |
|---|---|---|---|
| Elongation at Break | 5.5% | | ASTM D882 |
| Tensile Strength | 1.2 | kg | ASTM D882 |
| Weight | 50 | g/m² | |
| Density | 0.69 | g/m³ | |
| Dissolution time at 23° C. | 6 to 8 | s | IS 16154:2014 |

An example of the properties of a 65 μm thick paper-based liner (similar to the above but without a heat seal coating) that is suitable as a water-dispersible release liner are (at 23° C. and 50% relative humidity):

| Characteristic | Typical Values | | Measurement protocol |
|---|---|---|---|
| Elongation at Break | 2.7% | | ASTM D882 |
| Tensile Strength | 0.9 | kg | ASTM D882 |
| Weight | 30 | g/m² | |
| Density | 0.46 | g/m³ | |
| Dissolution time at 23° C. | 6 to 8 | s | IS 16154:2014 |

FIG. 3 shows a top view of the sanitary pad of FIG. 2a. The geometric shape of the sanitary pad 2 is an oval main body with opposing tabs 40 extending to either side of the main body. The opposing tabs 40 help secure the sanitary pad 2 to a surface with their positioning providing stability. Each tab 40 is substantially trapezoidal, with rounded corners. The opposing tabs 40 are removed by a distance C from the centre of the oval area. Alternatively the opposing tabs 40 are arranged centrally to the pad for a rotationally symmetrical shape. The tabs have a smaller inner length W than outer length X, reducing the length of the crease that occurs when the tabs are folded under the main body of the pad. This crease is often associated with unwanted fluid leakage; reducing the length of the crease acts to reduce the possibility of unwanted leakage.

The tabs may be formed of the top layer 10 and/or the leakproof layer 14 extending to either side of the main body. In case only one layer forms the tabs then the bond can go around the oval main body, whereas if both layers form the tabs then the bond can go around the perimeter of the tabs.

FIGS. 4a and 4b show a bottom and top view of a variant of the sanitary pad with a rectangular main body and tabs without rounded corners. In another variant the sanitary pad is formed of a main body, oval or rectangular or waisted or otherwise, with or without tabs.

In use, the sanitary pad absorbs menstrual fluid same as a conventional sanitary pad. After use the sanitary pad can be disposed for example in a toilet. On contact with water in the toilet the sanitary pad dissolves and with the hydraulic action it disperses. In particular the leakproof layer 14 dissolves and soluble binders (if included) in the top layer and absorbent layer dissolve and allow the other components of the layers (such as cellulosic fibres) to disperse. It is noteworthy that the leakproof layer 14 shows higher stability to blood than to water such as used in flushing. It is thought that this is due to the biological component of blood (platelets, red and white blood cells) and the volume of the solvent. The sanitary pad is dispersible enough that normal flushing action of a toilet disperses and dissolves it sufficiently for it to pass into the sewage system. Cellulosic and PVA and CMC components of the sanitary pad are biodegradable and can be processed by conventional sewage treatment.

In a variant the sanitary pad 2 includes a support layer. FIG. 5 shows a sanitary pad with a support layer 30 that is enclosed between the absorbent layer 12 and the leakproof layer 14. The support layer 30 can be a high density non-woven material. For dispersibility the support layer 30 can be of the same materials as described above with reference to the top layer 10. The support layer 30 can provide additional support to the sanitary pad. In an example a sanitary pad intended for night use (for use while sleeping) includes a support layer 30. For ease of manufacture the support layer 30 may carry (or be laminated or otherwise bonded to) a layer of super absorbent polymer that is functionally part of the absorbent layer 12 as described above. For dispersibility and biodegradability the support layer 30 is preferably a non-woven cellulosic material and can be of the same material as the top layer 10, as described in more detail above. In an example the support layer 30 is a wet-laid spunlace with a weight per unit area in the range of 50-75 gsm. Alternatively the support layer 30 is a pressed wood pulp sheet with a weight per unit area in the range of 10-60 gsm.

In a variant the leakproof layer 14 is folded over the top layer 10 to form a double seam. FIG. 6 shows a sanitary pad with a double seam 16 where the leakproof layer 14 encases top and bottom of a portion of the top layer 10. The double seam 16 can enable a stronger bond between the leakproof layer 14 and the top layer 10.

In a variant the absorbent layer 12 is not of uniform thickness but instead has an increased thickness in a central region. FIGS. 7 and 8 show a sanitary pad with an absorbent layer 12 that has a thick portion 50 in the centre of the pad. In use the thick portion 50 creates a raised area under the perineum to prevent back leaking, for example while sleeping. This can enable more efficient absorption. In the example illustrated in FIG. 8 the thick portion 50 viewed from above forms an elongated pentagon shape; other shapes are possible. In the illustrated example the absorbent layer 12 is about twice as thick at the thick portion 50 than in the surrounding portions; different proportions are possible. In an alternative a raised area is formed by a different layer, for example a portion of a support layer or a thickening insert layer.

In a variant the leakproof layer is a film that is applied to an adjacent layer such as the absorbent layer or a support layer or a bottom layer, for example by spray coating, laminating or co-extruding.

In another variant a surface of the leakproof layer is engineered to be hydrophobic (for example with a hydrophobic coating or by way of functionalisation of the surface with suitable hydrophobic functional groups). The hydrophobic surface is arranged facing the absorbent layer to assist fluid dispersion within the absorbent layer. As only one surface of the leakproof layer is hydrophobic, water-dispersibility and water-solubility of the layer can remain acceptable.

In a variant the sanitary pad includes an additional water-dispersible bottom layer. FIG. 9 shows a sectional view of a sanitary pad with a water-dispersible bottom layer 60. For dispersibility and biodegradability the bottom layer 60 is a non-woven cellulosic material and can be of the same material as the top layer 10, as described in more detail above, such as a wet-laid spunlace with a weight per unit area in the range of 50-75 gsm. The leakproof layer 14 is sandwiched between the top layer 14 and the bottom layer 60 and permits ultrasonic bonding of the top layer 14, the leakproof layer 14 and the bottom layer 60 at the edge 16 of the sanitary pad. The bottom layer 60 can provide better stability and strength of the sanitary pad in use, both mechanical (e.g. against warping) and against premature degradation of the leakproof layer (e.g. under the influence of perspiration).

For ease of manufacturing the sanitary pad the leakproof layer 14 can be provided laminated to the bottom layer 60. The leakproof layer 14 can be co-extruded with the bottom layer 60.

In the variant with a bottom layer the pressure sensitive adhesive is provided as described above but on the external surface of the bottom layer 60 instead of the leakproof layer 14.

FIG. 10 shows a sectional view of a sanitary pad with a bottom layer 60 that extends to form opposing tabs 40 as described above. In this variant the bottom layer 60 extend to either side of the main body beyond the bonded edge 16 of the top layer 14 and the leakproof layer 14. This arrangement can minimise the amount of material required to form tabs, while maintaining integrity of the sanitary pad.

In another variant the sanitary pad includes an additional water-dispersible acquisition distribution layer (ADL). A further suitable water-dispersible non-woven material can provide a suitable ADL, for example of the same material as the top layer 10 (e.g. a wet laid spunlace non-woven cellulose material), as described in more detail above. An ADL with a lower density (in gsm) than the top layer may be suitable. An ADL is preferably arranged between the top layer 10 and the absorbent layer 12. To improve lateral distribution of fluid the ADL is preferably with embossments.

FIGS. 13, 14 and 15 show examples of sanitary pads with a bottom layer as shown in FIG. 9 with an additional support layer 30 and/or acquisition distribution layer 70.

In a variant the top layer 10 is embossed to form a pattern on the top surface of the sanitary pad. FIG. 11 shows a top view of a pattern 70 for a top layer 10 of a sanitary pad. In the illustrated example the pattern 70 of embossments 74 is formed of small (edge lengths ca 0.5-2 mm) rectangles and rhomboids, the rectangles and rhomboids arranged in a pattern of diamonds (edge length around 15 mm) or overlapping hexagons covering the main surface of the pad. A variety of alternative patterns are suitable.

FIG. 12 shows a cross sectional view of a sanitary pad with embossments 74. The embossments 74 may extend across an ADL (if included) and at least partially into the absorbent layer 12, or they may extend across the absorbent layer 12 (and across a support layer if included) to the leakproof layer 14, as illustrated in FIG. 12. In this case a weak bond (such that the bond is severable without damage to the layers) may be formed between the top layer 10 and the leakproof layer 14 at the embossments 74. The embossments 74 in the top layer 10 can help reduce skin contact of fluid, and so give a dryer feel in use. The pattern 70 embossed in the top layer can help form channels to distribute fluid over the surface of the sanitary pad, enhancing absorption efficiency. The embossments 74 can also create crumple zones that enable the pad to be more flexible and less rigid during use. The embossments 74 can also help constrain movement of the underlying absorbent layer 12 by forming pockets of absorbent material and so help to avoid so-called core-slipping. Embossments in the top layer can provide better performance than perforations of the top layer, as perforations can reduce structural integrity of the sanitary pad unfavourably.

In an example embossment of the pattern in the top layer is performed by ultrasonic embossing simultaneously with ultrasonic bonding at the edge of the sanitary pad as described above. FIG. 11 shows an example of a tool for performing simultaneous embossing of the top layer with a pattern 70, and ultrasonic bonding at an edge 72 of the sanitary pad. In the illustrated example dimensions are indicated in mm. To achieve embossment the protrusions forming the embossed pattern 70 may be slightly recessed compared to the features forming the bonded edge 72. Alternatively both features may protrude equally, for example by 3 mm, but due to the presence of the absorbent layer 12 beneath the top layer 10 in the patterned area only a weak bond may be formed between the top layer 10 and the leakproof layer 14.

Suitable parameters for ultrasonic bonding and simultaneous embossing with the tool shown in FIG. 11 are for example:

Frequency—20 KHz

Energy—3000 Watt

Cycle Time—5 seconds

Amplitude—20 μm

Force—3000 Newton

Fixture Make—Aluminium (for ease of manufacturing) or titanium (for robustness)

Table 1 outlines some characteristics of some examples of materials for the various layers. Characteristics are determined according to testing protocols specified by the American Association of Textile Chemists and Colorists (AATCC), the International Standards Organisation (ISO) and the Bureau of Indian Standards (IS).

TABLE 1

| | | | | | | pH of |
|---|---|---|---|---|---|---|
| | | Perspiration | | Mass per | | aqueous |
| Layer | Composition | fastness | Absorbency | unit area | Thickness | extract |
| Test | | ISO 105- | AATCC-79- | IS1964 | Under 1 | |
| | | E04: 2013 | 14 | | kPa | |
| | | | | | pressure | |
| Units | | | Seconds | Grams per | mm | |
| | | | | square | | |
| | | | | metre | | |
| Top layer | Cellulosic | 4-5 | 1 | 54.3-57.8 | 0.42 | 6.5-7.5 |
| | nonwoven | | | | | |
| Top layer | Cellulosic | 4-5 | 1 | 61.7 | 0.46 | |
| | nonwoven | | | | | |
| Top layer | Nylon | 4-5 | 1 | 42.7 | — | 6.5-7.5 |
| | nonwoven | | | | | |
| Top layer | Viscose rayon | 4-5 | 1 | 31.3-34.6 | 0.2 | 6.5-7.5 |
| | nonwoven | | | | | |
| Absorbing | Viscose rayon | — | 1 | — | — | 6 |
| layer | fibres | | | | | |
| Absorbing | Cellulosic | — | 1 | — | — | 6.5-7.5 |
| layer | fibres | | | | | |
| Leakproof | Polyvinyl | — | — | 99.2 | 0.07 | 6.5-7.5 |
| layer | alcohol film | | | | | |
| Leakproof | Polyvinyl | — | — | 130 | 0.1 | 6.5-7.5 |
| layer | alcohol film | | | | | |

To test water-dispersibility and water-solubility of the sanitary pad, the UK SNAP protocol for testing disintegration in drainline (‘Test Protocol to determining the flushability of disposable products’, UKWIR Project WM07G202, March 2012) is performed. The test is performed as follows: a plain conical flask is attached to an orbital shaking table. A sample is introduced in the flask with tap water. The flask is agitated for 3 hours at 100 rpm. The content of the flask is then poured over a 12.5 mm perforated plate sieve (round holes) and the sieve is rinsed using a flow rate of 2 l/min for 1 minute. The level of disintegration is assessed by collecting the sample residuals retained by the sieve. The sample passes the test if greater than 50% of the sample mass (by dry weight) passes through the 12.5 mm perforated plate sieve and the pieces collected on the sieve are no more than 25 mm in their larger dimension.

Alternatively the UK “Fine to Flush: Specification for a testing methodology to determine whether a product is suitable for disposal through a drain or sewer system” (WIS 4-02-06, April 2019 Issue 1.1) Disintegration in the drainline test is performed, which is similar to the SNAP protocol summarised above. Key steps of the Fine to Flush protocol include:
Pour 1 litre of tap water into a baffled shake flask and add the sample.
Agitate the flask on an orbital shaker for 3 hours at 100 rpm.
Pour the contents of the flask onto a 12.5 mm sieve, inclined at 45°, taking care to spread the contents evenly over the plate.
Rinse the sample on the sieve in a 45° inclined position for 1 minute at ≤2 l/min flow rate, rotating the sieve 90° at 15 second intervals.
Collect the material retained on the upper surface and underside of the sieve and determine the proportion of material (by dry weight) passed through the sieve.

The sample is considered to disintegrate sufficiently if at least 50% of the sample mass passes through the 12.5 mm perforated plate sieve.

Alternatively the INDA EDANA “Guidelines for Assessing the Flushability of Disposable Nonwoven Products” (Edition 4, May 2018, Association of the Nonwoven Fabrics Industry (INDA); European Disposables and Nonwovens Association (EDANA)) Slosh Box Disintegration Test (4.2 FG502.R1(18)) is performed. Key steps include:
expose the sample to agitation in a box oscillating at 26 rpm with 2 litres of tap water for 60 minutes at 20° C.+/−3° C.;
pour the contents of the box onto a 12.5 mm perforated plate sieve resting at least 2 inches above a surface;
rinse for 120 seconds at 4 litres per minute flow rate;
collect the material retained on the sieve and determine the proportion of material (by dry weight) passed through the sieve.

The sample is considered to disintegrate sufficiently if more than 60% of the sample mass passes through the sieve for at least 80% of the individual replicates tested.

Alternatively the IWSFG “Publicly Available Specification (PAS) 3: 2018 Disintegration Test Methods—Slosh Box” (Jun. 5, 2018, International Water Services Flushability Group) Slosh Box Disintegration Test is performed. Key steps include:
expose the sample to agitation in a box oscillating at 18 rpm with 4 litres of tap water for 30 minutes at 15° C.+/−1° C.;
pour the contents of the box onto a 25 mm perforated plate sieve;
rinse for 60 seconds at 4 litres per minute flow rate;
collect the material retained on the sieve and determine the proportion of material (by dry weight) passed through the sieve.

The sample is considered to disintegrate sufficiently if more than 95% of the sample mass passes through the sieve.

To test absorption a glass cylinder with approximately 3 inches inner diameter (approximately 75 mm inner diameter) is placed on top of a sanitary pad, and 8 ml blood are poured onto the pad in the cylinder. The time for the blood to absorb into the pad is measured.

To test rewetting, once the absorption test described above is completed, a filter paper is placed on top of the sanitary pad. A 3 kg weight with a footprint of 10 cm by 5 cm is placed on the filter paper and removed after 3 minutes. The quantity of blood absorbed in the filter paper is determined by measuring the mass difference of the filter paper before and after the test.

To test absorption capacity the sanitary pad is immersed in saline solution for 10 minutes, drip dried for 2 minutes, and the absorption capacity is determined by measuring the mass difference of the sanitary pad before and after the test.

To test stability (leak proof performance), once the absorption test described above is completed, a filter paper is placed beneath the sanitary pad. A 3 kg weight with a footprint of 10 cm by 5 cm is placed on the sanitary pad and removed after 5 minutes (for immediate leakproof performance) or 24 hours (for longer term leakproof performance). The quantity of blood absorbed in the filter paper is determined by measuring the mass difference of the filter before and after the test.

To test biodegradability of the sanitary pad, the “Fine to Flush: Specification for a testing methodology to determine whether a product is suitable for disposal through a drain or sewer system” (WIS 4-02-06, April 2019 Issue 1.1) Appendix G ‘Determination of synthetic and non-synthetic organic components test’ is performed. The test is designed to dissolve natural organic material in a sample and leave synthetic organic material as a residue. The chemical dissolution provides a surrogate test to give information on the amount of natural biomass material (which can be assumed to be biodegradable) versus synthetic organic material (which may not be biodegradable). The test is performed as follows: a test sample is placed in a beaker in a fume hood and covered with 1000 ml of 14-15% sodium hypochlorite (NaClO) aqueous solution (bleach), then left for 24 hours, stirring at the beginning and end of the test period. The contents of the beaker are emptied onto a 0.5 mm sieve, and the sample is rinsed by pouring 2 litres of tap water from a 2 litre beaker over the sieve. The sample is considered biodegradable if no fibres or other visible residues remain on the sieve in 5 tests.

Alternatively the INDA EDANA “Guidelines for Assessing the Flushability of disposable Nonwoven Products” (Edition 4, May 2018, Association of the Nonwoven Fabrics industry (INDA); European Disposables and Nonwovens Association (EDANA)) Aerobic Biodisintegration/Biodegradation Tests are performed. The Aerobic Biodisintegration Test measures the total mass of a sample retained on a 1 mm sieve after being incubated with activated sludge for 14 days at ambient laboratory temperature. The average dry mass percent of material passing the sieve should exceed 95%. The Biodegradation Test follows the OECD 301B method (as adopted by the OECD on 17 Jul. 1992, Organisation for Economic Cooperation and Development) providing a standardised biodegradation test that measured the evolution of carbon dioxide resulting from biodegradation.

Alternatively one of the six OECD 301A-F test methods (as adopted by the OECD on 17 Jul. 1992, Organisation for Economic Cooperation and Development) are performed to determine biodegradability in an aerobic medium.

It will be understood that the present invention has been described above purely by way of example, and modifications of detail can be made within the scope of the invention. For example, sanitary pads may be provided in a wide variety of shape and sizes and intended uses, with or without tabs. While the present invention has been described in the context of a sanitary pad, the arrangement may be adapted for other water water-dispersible fluid absorbers, such as a wound dressing, an incontinence product, a diaper, a cleaning pad, or other uses where blood or other bodily fluids are taken up in a pad.

Reference numerals appearing in the claims are by way of illustration only and shall have no limiting effect on the scope of the claims. The term ‘comprising’ as used herein preferably means ‘including’.

1. A water-dispersible sanitary pad including:
a water-dispersible top layer;
a water-dispersible absorbent layer;
a leakproof layer of a polyvinyl alcohol; and
a water-dispersible bottom layer;
wherein the leakproof layer is bonded to the top layer and the bottom layer by ultrasonic welding.

2. A sanitary pad according to claim 1, wherein the absorbent layer is enclosed by the leakproof layer and the top layer.

3. A sanitary pad according to claim 1, wherein the top layer and/or the bottom layer is a cellulosic nonwoven fabric, preferably a wet-laid and/or spunlace and/or semi-hydrophobic cellulosic nonwoven fabric.

4. A sanitary pad according to claim 1, wherein the absorbent layer includes cellulosic fibres, preferably wood pulp fibres.

5. A sanitary pad according to claim 1, wherein the absorbent layer includes a super absorbent polymer.

6. (canceled)

7. A sanitary pad according to claim 5, wherein the super absorbent polymer is bonded to an adjacent material or layer.

8. A sanitary pad according to claim 7, wherein the super absorbent polymer is bonded to the leakproof layer.

9. (canceled)

10. A sanitary pad according to claim 1, wherein the sanitary pad is biodegradable.

11. A sanitary pad according to claim 1, further including a water-dispersible acquisition distribution layer, preferably arranged between the top layer and the absorbent layer.

12. A sanitary pad according to claim 11, wherein the acquisition distribution layer is a cellulosic nonwoven fabric, preferably a wet-laid and/or spunlace cellulosic nonwoven fabric.

13. A sanitary pad according to claim 1, further including a water-dispersible support layer, preferably arranged between the absorbent layer and the leakproof layer.

14. A sanitary pad according to claim 13, wherein the support layer is a cellulosic nonwoven fabric, preferably a wet-laid and/or spunlace cellulosic nonwoven fabric.

15. A sanitary pad according to claim 1, wherein the leakproof layer is a polyvinyl alcohol film, preferably between 20 and 150 μm thick, more preferably between 30 and 90 μm thick, yet more preferably between 30 and 65 μm thick.

16. A sanitary pad according to claim 1, wherein the leakproof layer is laminated to the bottom layer and/or co-extruded with the bottom layer.

17. A sanitary pad according to claim 1, wherein the top layer and optionally the absorbent layer includes embossments, preferably formed by ultrasonic embossing.

18. A sanitary pad according to claim 17, wherein the top layer is weakly bonded to the leakproof layer at the embossments.

19-26. (canceled)

27. A method of manufacturing a water-dispersible sanitary pad including:
assembling a water-dispersible top layer, a water-dispersible absorbent layer, a leakproof layer of a polyvinyl alcohol and a water-dispersible bottom layer; and
bonding the leakproof layer to the top layer and the bottom layer by ultrasonic welding.

28. A method according to claim 27, further comprising embossing one or more layers by ultrasonic embossing.

29. A method according to claim 27, further comprising
enclosing the absorbent layer between the leakproof layer and the top layer.

30-37. (canceled)

38. A water-dispersible sanitary pad including:
a water-dispersible top layer;
a water-dispersible absorbent layer; and
a water-dispersible leakproof layer;
wherein the absorbent layer comprises a cellulose-based super absorbent material, an alginate-based super absorbent material, a chitosan-based super absorbent material, and/or a polyethylene glycol-based super absorbent material.

39-44. (canceled)
